(19) United States
(12) Patent Application Publication
(10) Pub. No.: US 2013/0273081 A1
(43) Pub. Date: Oct. 17, 2013

(54) VACCINATION AGAINST PCSKK 9 FOR LOWERING CHOLESTEROL

(71) Applicants: Paolo Monaci, Rome (IT); Andrea Carfi, Rome (IT)

(72) Inventors: Paolo Monaci, Rome (IT); Andrea Carfi, Rome (IT)

(21) Appl. No.: 13/637,144

(22) PCT Filed: Mar. 25, 2011
(86) PCT No.: PCT/EP11/54646

§ 371 Date: May 30, 2013

(30) Foreign Application Priority Data
Mar. 25, 2010 (GB) 1005005.2

(51) Int. Cl. A61K 38/48

(52) U.S. Cl. CPC A61K 38/482
USPC 424/184.1; 536/23.2; 435/226

(57) ABSTRACT

The present invention relates to pharmaceutical compositions comprising PCSK9 DNA or PCSK9 proteins or PC-SK9 peptides and CpG adjuvant; this composition is used to lower cholesterol levels specifically LDL cholesterol levels.

### FIELD OF THE INVENTION

The present application relates generally to an anti-hPCSK9 vaccine useful in lowering of Low Density Lipoprotein cholesterol (LDL-c) for use in a method of treatment of the human body by therapy. More specifically the anti-hPCSK9 vaccine is useful in the treating and preventing diseases associated with increased levels of circulating LDL-c.

### BACKGROUND

Atherosclerosis and its clinical consequences, coronary heart disease (CHD), stroke and peripheral vascular disease, represent a truly enormous burden to the health care systems of the industrialized world. In the United States alone, approximately 13 million patients have been diagnosed with coronary heart disease, and greater than one half million deaths are attributed to coronary heart disease each year. Further, this toll is expected to grow over the next quarter century as an epidemic in obesity and diabetes continues to grow.

It has long been recognized that in mammals, variations in circulating lipoprotein profiles correlate with the risk of atherosclerosis and coronary heart disease. The clinical success of HMG-CoA Reductase inhibitors, especially the statins, in reducing coronary events is based on the reduction of circulating LDL-c), levels of which correlate directly with increased risk for atherosclerosis. More recently, epidemiologic studies have demonstrated an inverse relationship between High Density Lipoprotein cholesterol (HDL-c) levels and atherosclerosis, leading to the conclusion that low serum HDL-c levels are associated with an increased risk for coronary heart disease.

Pro-protein convertase subtilisin-like/kexin type 9 (PCSK9) has recently emerged as a key determinant of liver low-density lipoprotein receptor (LDLR) and LDL-c plasma levels, and consequently of cardiovascular health in humans PCSK9 belongs to the mammalian pro-protein convertase family of serine proteases and is expressed predominantly in the liver and small intestine. Following auto-cleavage in the Endoplasmic Reticulum the pro-protein is secreted into the plasma in an auto-inhibited form lacking enzymatic activity. The addition of PCSK9 to cultured cell medium has been shown to result in LDLR degradation both overall and at the cell surface, and in decreased LDL-c uptake. Consistently, several groups have reported that PCSK9 binds the LDLR ectodomain. Furthermore, prior to degradation, the PCSK9/LDLR complex is internalized in a manner dependent on the association of a cytosolic region of the receptor with Autosomal Recessive Hypercholesterolemia (ARH), an adaptor protein required for LDLR endocytosis in liver cells. Together, these data are consistent with a mechanism whereby PCSK9 acts as a chaperone by binding LDLR and shuttling the receptor to lysosomes for degradation.

PCSK9 has recently emerged as a central factor in the regulation of hepatic LDLR levels and plasma LDL-c catabolism. Nonsense mutations in human PCSK9 have been reported that are associated with reduced plasma levels of LDL-c and reduced risk of coronary heart disease. Genetic KO or silencing of the PCSK9 gene by antisense oligonucleotides or siRNA resulted in increased levels of hepatic LDLR and accelerated removal of cholesterol from the plasma in rodents and/or non-human primates. Further, although in vitro experiments have shown that disruption of the interaction between secreted PCSK9 and LDLR by antibodies or LDLR-EGF(AB) fragments can partially restore LDL-c uptake and LDLR cell surface levels in liver cells expressing PCSK9 (Duff et al, Biochem J., 419, 577-584 (2009)). Finally, a strong decrease in LDL-c levels has been observed in non-human primates and mice treated with an antibody against PCSK9 that competes with LDLR binding.

An immunological-knock down (IKD) method is an approach for the in vivo validation and functional study of endogenous gene products. This method relies on the ability to elicit a transient humoral response against the selected endogenous target protein. Anti-target antibodies specifically bind to the target protein and effectively neutralize its activity.

Despite the significant therapeutic advance that statins such as simvastatin (ZOCOR®) represent, statins only achieve a risk reduction of approximately one-third in the treatment and prevention of atherosclerosis and ensuing atherosclerotic disease events.

The present invention provides: (i) DNA encoding xenogenic PCSK9 or optionally mutated xenogenic PCSK9; (ii) DNA encoding optionally mutated human-non human chimeric PCSK9; (iii) DNA encoding mutated human PCSK9; (iv) an immunologically active fragment of (i) or ((ii); (v) an immunologically active and mutation containing fragment of (iii) or (iv); (vi) a protein embodied by any one of (i) to (v); for use in a method of treatment of the human body by therapy. The molecules used in the present invention have the potential to increase liver LDL receptor levels and reduce plasma LDL-c levels.

PCSK9 is the ninth member of the subtilisin family of kexin-like proconvertases that have already been identified. Catalytic domain of non-human species is to be numbered analogously. That is to say, the catalytic domain is the position following the prodomain. Similarly to other PCSK9 family members, it contains a signal sequence (amino acids 1-30) followed by a prodomain (amino acids 31-152) and the catalytic domain (amino acids 152-425). The use of DNA or protein providing an immunologically active portion of the catalytic domain is preferred. PCSK9 lacks the classical P-domain which is required for folding and regulation of protease activity in other proprotein convertases. In this case the catalytic domain is followed by a 278-amino acid cysteine- and histidine-rich C-terminal region.

The present invention, furthermore, provides for compositions, recombinant protein sequences, encoding nucleic acid sequences, vectors, host cells, and methods of employing the foregoing which comprise, encode a protein which comprises, or utilize fragments of the disclosed consensus sequences. “Fragments” as defined herein refer to fragments of a consensus sequence (nucleotide or protein) which are capable of eliciting an immune response such as anti-PCSK9 antibodies (as determined by various cellular assays available and widely appreciated by the skilled artisan; for purposes of exemplification and not limitation). The sequence of the fragment or sequence comprising the fragment should hybridize under stringent conditions to the complement of at least one natural antigen sequence from which it was derived (directly or indirectly). Methods for hybridizing nucleic acids are well-known in the art; see, e.g. Ausubel, Current Protocols in Molecular Biology, John Wiley & Sons, N.Y., 6.3.1-6.3.6, 1989. For purposes of exemplification and not limitation, moderately stringent hybridization conditions may, in specific embodiments, use a prewashing solution containing 5× sodium chloride/sodium citrate (SSC), 0.5% w/v SDS, 1.0 mM EDTA (pH 8.0), hybridization buffer of about 50% v/v formamide, 6×SSC, and a hybridization temperature of 55° C. (or other similar hybridization solutions, such as one containing about 50% v/v formamide, with a hybridization temperature of 42° C.), and washing conditions of 60° C., in 0.5×SSC, 0.1% w/v SDS. For purposes of exemplification and not limitation, stringent hybridization conditions may, in specific embodiments, use the following conditions: 6×SSC at 45° C., followed by one or more washes in 0.1×SSC, 0.2% SDS at 68° C. One of skill in the art may, furthermore, manipulate the hybridization and/or washing conditions to increase or decrease the stringency of hybridization such that nucleic acids comprising nucleotide sequences that are at least 80, 85, 90, 95, 98, or 99% identical to each other typically remain hybridized to each other. The basic parameters affecting the choice of hybridization conditions and guidance for devising suitable conditions are set forth by Sambrook et al., Molecular Cloning: A Laboratory Manual, Cold Spring Harbor Laboratory Press, Cold Spring Harbor, N.Y., chapters 9 and 11, 1989 and Ausubel et al. (eds), Current Protocols in Molecluar Biology, John Wiley & Sons, Inc., sections 2.10 and 6.3-6.4, 1995. Such parameters can be readily determined by those having ordinary skill in the art based on, for example, the length and/or base composition of the DNA.

The fragments, in specific embodiments, comprise a string of amino acids selected from the group consisting of: (1) amino acids 1-16; (2) amino acids 9-24; (3) amino acids 17-32; (4) amino acids 25-40; (5) amino acids 33-48; (6) amino acids 41-56; (7) amino acids 49-64; (8) amino acids 57-72; (9) amino acids 65-80; (10) amino acids 73-88; (11) amino acids 81-96; (12) amino acids 89-104; (13) amino acids 97-112; (14) amino acids 105-120; (15) amino acids 113-128; (16) amino acids 121-136.

An immunologically active fragment is defined as a fragment which can induce an immune response in vivo. The immune response is measured as the creation of anti-hPCSK9 antibodies in vivo in response to the immunologically active fragment.

A fragment is defined within the invention as a piece of DNA encoding a protein having the length between 20 and 700 amino acids. Preferably a fragment is a piece of DNA encoding a protein having the length between 20 and 500 amino acids. More preferably a fragment is a piece of DNA encoding a protein having the length between 20 and 300 amino acids. Most preferably a fragment is a piece of DNA encoding a protein having the length between 20 and 280 amino acids. A fragment is defined within the invention as a piece of DNA encoding a protein having the length between 20 and 200 amino acids.

One aspect of the invention provides that the DNA and/or protein may be created using Fusion technology. The DNA and/or proteins created through the joining of two or more genes which originally coded for separate proteins. Translation of this chimeric gene results in a single polypeptide. In this case the chimeric DNA and/or protein may be created from more than one xenogenic PCSK9. Preferably the DNA and/or protein of the present invention contain PCSK9 from three species and more preferably two PCSK9 species.

In another aspect of this invention the DNA and/or protein of the present invention may be created using human and non-human PCSK9. In order for the DNA and/or protein to be considered chimeric the fragment is defined within the invention as a piece of DNA encoding a protein wherein at least five amino acids are from a different species. Preferable in order for the DNA and/or protein to be considered chimeric the fragment is defined within the invention as a piece of DNA encoding a protein wherein at least five amino acids are from a different species. Most preferably in order for the DNA and/or protein to be considered chimeric the fragment is defined within the invention as a piece of DNA encoding a protein wherein at least one amino acid is from a different species.

An object of the present invention is to provide a vaccine antigen which elicits an immune response against hPCSK9 wherein this immune response reduces the level of plasma LDL-c in the patient. This immune response can be measured by the standard techniques such as an effect elicited in vivo on a non-human animal model or human. It can also be detected from the presence of antibodies to hPCSK9. A well know tool used to detect the presence of antibody or an antigen in a sample is Enzyme Linked Immunosorbent Assay (ELISA) also know as Enzyme immunoassay or EIA.

The object of this invention is to force the body to create an immune response to hPCSK9 which would not normally be found within the body. Therefore any detection of antibodies created against hPCSK9 can be indicative that an immune response has been created. Therefore a man skilled in the art would recognise that detection of anti-hPCSK9 antibodies in vivo is indicative of an immune response. Preferably immunological activity is measured by the presence of anti-hPCSK9 antibodies. More preferably immunological activity is measured by the presence of anti-hPCSK9 antibodies as determined using ELISA. More preferable immunological activity is measured by the presence of anti-hPCSK9 antibodies found in serum. More preferable immunological activity is measured by the presence of anti-hPCSK9 antibodies found in serum as determined using ELISA. Most preferably immunological activity is measured by the presence of anti-hPCSK9 antibodies within the serum 14 days after the first injection of DNA and/or protein of this invention. Most preferably immunological activity is measured by the presence of anti-hPCSK9 antibodies within the serum at least 10-60 days after the first injection of DNA and/or protein of this invention determined by ELISA.

The present invention further provides that more than one DNA and/or protein as defined therein may be administered. The catalytic domain of PCSK9 is derived from a xenogenic animal PCSK9, preferably derived from non-human mammal or primate PCSK9, more preferably derived from rodent PCSK9 and rabbit PCSK9, canine PCSK9, cat PCSK9, rhesus monkey PCSK9, macaque PCSK9, marmoset PCSK9, tamarin PCSK9, spider monkey PCSK9, owl monkey PCSK9, vervet monkey PCSK9, squirrel monkey PCSK9, and baboon PCSK9. Primates would also include great ape PCSK9, such as gorilla PCSK9, chimpanzee PCSK9, and orangutan PCSK9.

More preferably the catalytic domain of PCSK9 is derived from canine PCSK9 rodent PCSK9 or rhesus monkey PCSK9 and most preferably derived from mouse PCSK9.

The invention provides that the DNA and/or protein can be used for treating or preventing diseases associated with high LDL-cholesterol such as cardiovascular disease and atherosclerosis. In one aspect DNA and protein from the present invention is combined to create an immune response to hPCSK9. In another aspect of the present invention DNA from the present invention is used to create an immune response to hPCSK9. In a further aspect of the present invention protein from the present invention is used to create the immune response to hPCSK9.

Diseases or conditions that may be treated with compounds of this invention, or which the patient may have a reduced risk of developing as a result of being treated with the compounds of this invention, include: atherosclerosis, peripheral vascular disease, dyslipidemia, hyperbetalipoproteinemia, hypoalphalipoproteinemia, hypercholesterolemia, hypertriglyceridemia, familial-hypercholesterolemia, cardiovascular disorders, angina, ischemia, cardiac ischemia, stroke, myocardial infarction, reperfusion injury, angioplastic restenosis, hypertension, vascular complications of diabetes, obesity, endotoxemia, and metabolic syndrome.

The present invention discloses a method of treatment of a subject suffering from a disease associated with elevated PCSK9 which comprises administering to that subject a therapeutically effective amount of (i) DNA encoding mutated human or optionally mutated xenogenic PCSK9; (ii) DNA encoding optionally mutated human-non human chimera PCSK9; (iii) DNA encoding mutated human PCSK9; (iv) an immunologically active fragment of (i) or ((ii); (v) an immunologically active and mutation containing fragment of (iii) or (iv); (vi) a protein embodied by any one of (i) to (v); to that subject. In particular the method is for the treatment of diseases associated with cholesterol. Further provided within this invention is a method for treating cardiovascular diseases such as, atherosclerosis, peripheral vascular disease, dyslipidemia, hyperbetalipoproteinemia, hypoalphalipoproteinemia, hypercholesterolemia, hypertriglyceridemia, familial-hypercholesterolemia, cardiovascular disorders, angina, ischemia, cardiac ischemia, stroke, myocardial infarction, reperfusion injury, angioplastic restenosis, hypertension, vascular complications of diabetes, obesity, endotoxemia, or metabolic syndrome.

Another object of the invention relates to a method of treating an individual comprising administering to an individual an amount of DNA and/or protein of this invention capable of eliciting from the individual a B- or T-cell immune response effective to prevent or to decrease the diseases associated with high plasma LDL-cholesterol.

The invention also relates to methods for the treatment of diseases associated with high LDL-c in an individual comprising administering to an individual in need of such treatment an amount of DNA and/or protein of this invention effective to decrease the level of LDL-c present.

The invention also relates to antibodies which immunoreact with hPCSK9 and/or compositions thereof.

It is an additional object of this invention to provide DNA and/or protein which is capable to induce or elicit serum antibodies which have activity against hPCSK9. DNA and/or proteins of this invention are effective as vaccines to induce serum antibodies which are useful to lower the levels of LDL-c.

This invention also relates to various suitable expression systems, viral particles, vectors, vector system, and transformed host cells containing those nucleic acids.

A patient is a human or mammal, and is most often a human. A “therapeutically effective amount” is the amount of compound that is effective in obtaining a desired clinical outcome in the treatment of a specific disease.

Suitably DNA and/or protein derived from the invention will be administered in the range of 0.1-1000 μg, preferably 10-100 μg, especially 25-75 μg and for example 50 μg per dose.

### Vaccine Composition

Co-administration of vaccines with compounds that can enhance the immune response against the antigen of interest, known as adjuvants, has been extensively studied. In addition to increasing the immune response against the antigen of interest, some adjuvants may be used to decrease the amount of antigen necessary to provoke the desired immune response or decrease the number of injections needed in a clinical regimen to induce a durable immune response and provide protection from disease. The present invention may contain a pharmaceutically acceptable carrier, such as excipient, dilutent, stabilizer, buffer or alternative substance that is designed to facilitate administration of the DNA and/or protein in the desired amount to the patient. The composition may also contain additionally physiologically acceptable composnents, such as buffer, normal saline or phosphate buffered saline, sucrose, other salts and/or polysorbate.

### CpG

“Immunostimulatory oligonucleotides containing unmethylated CpG dinucleotides (“CpG”) are known in the art as being adjuvants when administered by both systemic and mucosal routes (WO 96/02555, EP 468520, Davis et al., J.Immunol., 1998, 160(2):870-876; McCluskie and Davis, J. Immunol., 1998, 161(9):4463-6). CpG is an abbreviation for cytosine-guanosine dinucleotide motifs present in DNA. Historically, it was observed that the DNA fraction of BCG could exert an anti-tumour effect. In further studies, synthetic oligonucleotides derived from BCG gene sequences were shown to be capable of inducing immunostimulatory effects (both in vitro and in vivo). It has been concluded that certain palindromic sequences, including a central CG motif, carried this activity. The central role of the CG motif in immunostimulation was later elucidated in a publication in Krieg, Nature 374, p 546 1995. Detailed analysis has shown that the CG motif has to be in a certain sequence context, and that such sequences are common in bacterial DNA but are rare in vertebrate DNA. The immunostimulatory sequence is often: Purine, Purine, C, G, pyrimidine, pyrimidine; wherein the CG motif is not methylated, but other unmethylated CpG sequences are known to be immunostimulatory and may be used in the present invention.

In certain combinations of the six nucleotides a palindromic sequence is present. Several of these motifs, either as repeats of one motif or a combination of different motifs, can be present in the same oligonucleotide. The presence of one or more of these immunostimulatory sequence containing oligonucleotides can activate various immune subsets, including natural killer cells (which produce interferon γ and have cytolytic activity) and macrophages (Woodridge et al Vol. 89 (no. 8), 1977). Although other unmethylated CpG containing sequences not having this consensus sequence have now been shown to be immunomodulatory.

CpG when formulated into vaccines, is generally administered in free solution together with free antigen (WO 96/02555; McCluskie and Davis, supra) or covalently conjugated to an antigen (PCT Publication No. WO 98/16247), or formulated with a carrier such as aluminium hydroxide ((Hepatitis surface antigen) Davis et al. supra; Brazolot-Millan et al., Proc. Natl. Acad. Sci., USA, 1998, 95(26), 15553-8).”

Suitably CpG will be presenting the range of 0.1 μg per dose to 1000 μg, preferably 10-100 μg especially 25-75 μg and for example 50 μg per dose.

### Aluminium Adjuvant

Aluminium has long been shown to stimulate the immune response against co-administered antigens, primarily by stimulating a T_{H}2 response. In addition to HPV VLPs and an ISCOM-type adjuvant, the formulations of this aspect of the present invention are adsorbed to aluminium adjuvant. It is preferred that the aluminium adjuvant of the compositions provided herein is not in the form of an aluminium precipitate. Aluminium-precipitated vaccines may increase the immune response to a target antigen, but have been shown to be highly heterogeneous preparations and have had inconsistent results (see Lindblad E. B. Immunology and Cell Biology 82: 497-505 (2004)). Aluminium-adsorbed vaccines, in contrast, can be preformed in a standardized manner, which is an essential characteristic of vaccine preparations for administration into humans. Moreover, it is thought that physical adsorption of a desired antigen onto the aluminium adjuvant has an important role in adjuvant function, perhaps in part by allowing a slower clearing from the injection site or by allowing a more efficient uptake of antigen by antigen presenting cells.

The aluminium adjuvant of the present invention may be in the form of aluminium hydroxide (Al(OH)₃), aluminium phosphate (AlPO₄), aluminium hydroxyphosphate, amorphous aluminium hydroxyphosphate sulfate (AAHS) or so-called “alum” (KAl(SO₄).12H₂O) (see Klein et al., Analysis of aluminium hydroxyphosphate vaccine adjuvants by (27) A1 MAS NMR., J. Pharm. Sci. 89(3): 311-21 (2000)). In exemplary embodiments of the invention provided herein, the aluminium adjuvant is aluminium hydroxyphosphate or AAHS. The ratio of phosphate to aluminium in the aluminium adjuvant can range from 0 to 1.3. In preferred embodiments of this aspect of the invention, the phosphate to aluminium ratio is within the range of 0.1 to 0.70. In particularly preferred embodiments, the phosphate to aluminium ratio is within the range of 0.2 to 0.50.

In some embodiments of the invention, the aluminium adjuvant is in the form of AAHS (referred to interchangeably herein as Merck aluminium adjuvant (MAA)). MAA carries zero charge at neutral pH, while AlOH carries a net positive charge and AlPO₄ typically carries a net negative charge at neutral pH. MAA has a higher capacity to bind HPV VLPs than AlOH. In addition, VLPs adsorbed to MAA can induce a greater humoral immune response in mice than VLPs adsorbed to AlOH. Caulfield et al., Human Vaccines 3: 139-146 (2007). While not wishing to be bound by theory, it is possible that net charge of the aluminium adjuvant can affect its ability to bind the VLP antigen, with strongly charged adjuvants unable to bind antigen as strongly as neutral charged adjuvants. For this reason, it is preferred that the aluminium adjuvant of the pharmaceutical compositions of the present invention have zero point surface charge at neutral pH. One of skill in the art will be able to vary the buffer, salt concentration and/or percent of free phosphate in order to allow a zero point surface charge at neutral pH.

One of skill in the art will be able to determine an optimal dosage of aluminium adjuvant that is both safe and effective at increasing the immune response to the targeted HPV type(s). For a discussion of the safety profile of aluminium, as well as amounts of aluminium included in FDA-licensed vaccines, see Baylor et al., Vaccine 20: S18-S23 (2002). Generally, an effective and safe dose of aluminium adjuvant varies from 150 to 600 μg/dose (300 to 1200 μg/mL concentration). In specific embodiments of the formulations and compositions of the present invention, there is between 200 and 300 μg aluminium adjuvant per dose of vaccine. In alternative embodiments of the formulations and compositions of the present invention, there is between 300 and 500 μg aluminium adjuvant per dose of vaccine.

### ISCOM Adjuvant

An “ISCOM-type adjuvant” is an adjuvant comprising an immune stimulating complex (ISCOM), which is comprised of a saponin, cholesterol, and a phospholipid, which together form a characteristic caged-like particle, having a unique spherical, caged-like structure that contributes to its function (for review, see Barr and Mitchell, Immunology and Cell Biology 74: 8-25 (1996)). This term includes both ISCOM adjuvants, which are produced with an antigen and comprise antigen within the ISCOM particle and ISCOM matrix adjuvants, which hollow ISCOM-type adjuvants that are produced without antigen. In preferred embodiments of the compositions and methods provided herein, the ISCOM-type adjuvant is an ISCOM matrix particle adjuvant, such as ISCOMATRIX®, which is manufactured without antigen (ISCOM® and ISCOMATRIX® are the registered trademarks of CSL Limited, Parkville, Australia).

### ISCOM and Aluminium

The ISCOM-type adjuvant comprises a saponin, cholesterol, and a phospholipid, and forms an immune-stimulating complex or ISCOM. The potent adjuvant activity of saponins, which are typically isolated from the bark of the Quillaia saponaria tree, was first documented over 80 years ago (for review, see Barr and Mitchell, Immunology and Cell Biology 74: 8-25 (1996); and Skene and Sutton, Methods 40: 53-59 (2006)). Compared to aluminium adjuvants, ISCOM-type adjuvants or ISCOMs are able to provoke a broader immune response to a co-administered antigen, comprising both T-cell and antibody responses. However, a potential for toxicity and haemolytic activity was found, limiting the promise of saponins for human or animal use at that time.

Since then, it was discovered that saponins, when combined with cholesterol and phospholipid, form a characteristic particle having a caged-like structure comprised of twenty or more subunits. This unique structure contributes to the adjuvant activity of the ISCOMs. Additionally, the incorporation of saponins into ISCOMs, together with cholesterol and phospholipid, was shown to eliminate the haemolytic activity of saponins. It was also shown that less adjuvant was needed to induce an immune response when ISCOMs were utilized as adjuvant compared to free saponins (see Skene and Sutton, supra). For these reasons, ISCOMs have been intensely studied as potential vaccine adjuvants.

### Routes of Administration

### Vaccine Compositions

Vaccine compositions of the present invention may be used alone at appropriate dosages which allow for optimal inhibition of the endogenous circulating PCSK9 with minimal potential toxicity. In addition, co-administration or sequential administration of other agents may be desirable.

The formulations and compositions of the present invention may be administered to a patient by intramuscular injection, subcutaneous injection, intradermal introduction, or impression though the skin. Other modes of administration such as intraperitoneal, intravenous, or inhalation delivery are also contemplated. In preferred embodiments of the invention, the vaccines and pharmaceutical compositions are administered by intramuscular administration.

In some embodiments of this invention, the pharmaceutical compositions and formulations disclosed herein are administered to a patient in various prime/boost combinations in order to induce an enhanced, durable, immune response. In this case, two pharmaceutical compositions are administered in a “prime and boost” regimen. For example the first composition is administered one or more times, then after a predetermined amount of time, for example, 2 weeks, 1 month, 2 months, six months, or other appropriate interval, a second composition is administered one or more times.

Preferably, the pharmaceutical compositions used in a clinical regimen comprise PCSK9 of the same type or combination of types. However, it may also be desirable to follow a clinical regimen in which two different PCSK9 pharmaceutical compositions are administered to a patient with an appropriate interval of time separating the two vaccine administrations. For example, a vaccine composition comprising DNA PCSK9 may be administered at one point in time, followed by a PCSK9 vaccine composition comprising protein PCSK9 at a second point in time, after a pre-determined length of time has passed. In such cases, each of the two different PCSK9 vaccine compositions may be administered to the patient once, or more than one time, separated by an appropriate length of time.

In accordance with one aspect of the present invention, it was shown that that a two-dose clinical regimen using a DNA or protein PCSK9 vaccine adjuvanted with CpG can induce an immune response.

In that respect, the present invention provides a method of raising anti-hPCSK9 antibodies in humans which will selectively inactivate the secreted endogenous h-PCSK9 lowering LDL-c levels in a human patient for use in a method of treatment of the human body by therapy comprising: (a) introducing into the patient a first pharmaceutical composition comprising protein PCSK9 and/or DNA PCSK9, CpG adjuvant; (b) allowing a predetermined amount of time to pass; and (c) introducing into the patient a second pharmaceutical composition comprising DNA PCSK9 and/or protein PCSK9 and a CpG adjuvant to the patient.

In specific embodiments of the method described above, the first and second compositions are the same and the clinical regimen includes at least one injection of the composition to “prime” the immune response to hPCSK9 and at least one injection to “boost” the immune response. However, other methods in which multiple injections to prime and/or boost the immune response are also contemplated by the invention described herein.

In some circumstances, it may be desirable to provide a multi-dose PCSK9 vaccine formulation which comprises more than one dose of vaccine in the same vial. If a multi-dose formulation is desired, an anti-microbial preservative should be used to kill or prevent the growth of microorganisms, such as bacteria and fungi. Multi-dose vaccine formulations containing anti-microbial preservatives provide several advantages over single dose formulations, including allowing multiple doses of vaccine to be withdrawn from the vial over a period of time without the concern that the first withdrawal inadvertently introduced microbial contamination (Meyer et al., J. Pharm. Sci. 96(12): 3155-3167 (2007)). Many marketed vaccine products, which are unrelated to HPV, comprise phenoxyethanol (DAPTACEL® (Sanofi Pasteur, Lyon, France), PEDIARIX®, INFANRIX®, HAVRIX®, and TWINRIX® (GlaxoSmithKline (GSK), Brentford, Middlesex, United Kingdom) or thimerosal (PEDIARIX® and ENGERIX-B® (GSK)) as anti-microbial preservatives (see Meyer et al., supra). In addition PNEUMOVAX® 23 (Merck & Co., Inc., Whitehouse Station, N.J.) formulations contain phenol as an antimicrobial preservative. However, the compatibility of DNA and/or protein PCSK9-containing vaccine formulations with anti-microbial preservatives has not been previously addressed.

Thus, in accordance with one aspect of the present invention, it was shown that the addition of an antimicrobial preservative selected from the group consisting of: m-cresol, phenol, and benzyl alcohol, to vaccine formulations comprising DNA and/or protein PCSK9 is effective at reducing or eliminating microbes and does not negatively impact the structural and thermal stability of the DNA and/or protein PCSK9 at 2-8° C. Thus, the invention also relates to DNA and/or protein PCSK9 vaccine formulations comprising DNA and/or protein PCSK9 and an antimicrobial preservative selected from the group consisting of: m-cresol, phenol and benzyl alcohol. The vaccine formulations according to this aspect of the invention may also include CpG adjuvant and ISCOM-type adjuvant and an aluminium adjuvant, as described above.

In some preferred embodiments of this aspect of the invention, m-cresol is included in the multi-dose PCSK9 vaccine formulation at a concentration of about 0.15 to about 0.31%. In more preferred embodiments, the multi-dose vaccine formulations comprise m-cresol at a concentration of about 0.25 to about 0.31%. In one preferred embodiment, m-cresol is included in the multi-dose formulation at a concentration of about 0.3%.

In alternative embodiments of the invention, phenol is included in the multi-dose PCSK9 vaccine formulations at a concentration of about 0.25 to about 0.55%. In more preferred embodiments, phenol is included at a concentration of about 0.4 to about 0.55%. In one particularly preferred embodiment, a multi-dose PCSK9 vaccine formulation comprising phenol at a concentration of about 0.5% is provided.

In still further embodiments, the multi-dose PCSK9 vaccine formulations comprise benzyl alcohol at a concentration of about 0.75 to about 1.2%. In more preferred embodiments, benzyl alcohol is included in the multi-dose vaccine formulation at a concentration from about 0.8% to about 1.0%. In a particularly preferred embodiment of this aspect of the invention, the concentration of benzyl alcohol is 0.9%.

Accordingly, one aspect of the present invention relates to a multi-dose anti-hPCSK9 vaccine formulation comprising: (a) DNA and/or protein PCSK9 of at least one DNA and/or protein PCSK9 type, wherein the DNA and/or protein PCSK9 type is selected from the group consisting of: (i) DNA encoding wild type or mutated xenogenic PCSK9; (ii) DNA encoding optionally mutated human-non human chimera PCSK9; (iii) DNA encoding mutated human PCSK9; (iv) an immunologically active fragment of (i) or ((ii); (v) an immunologically active and mutation containing fragment of (iii) or (iv); (vi) a protein embodied by any one of (i) to (v); (b) a CpG adjuvant; and (c) an anti-microbial preservative selected from the group consisting of: m-cresol, phenol and benzyl alcohol; wherein said DNA and/or protein PCSK9 are adsorbed onto said CpG adjuvant.

The multi-dose anti-hPCSK9 vaccine formulation described above may optionally include an ISCOM-type adjuvant.

### Combination Therapy

Compounds of the invention may be used in combination with other drugs that may also be useful in the treatment or amelioration of the diseases or conditions for which DNA and/or protein of the present invention are useful. Such other drugs may be administered, by a route and in an amount commonly used therefor, contemporaneously or sequentially with DNA and/or protein of the present invention. When DNA and/or protein of the present invention is used in combination with one or more other drugs, a pharmaceutical composition in unit dosage form containing such other drugs and the DNA and/or protein of the present invention is preferred. However, the combination therapy also includes therapies in which the DNA and/or protein of the present invention and one or more other drugs are administered on different schedules.

It is also contemplated that when used in combination with one or more other active ingredients, the DNA and/or protein of the present invention and the other active ingredients may be used in lower doses than when each is used singly. Accordingly, the pharmaceutical compositions of the present invention include those that contain one or more other active ingredients, in addition to DNA and/or protein of the present invention.

Examples of other active ingredients that may be administered in combination with DNA and/or protein of the present invention, and either administered separately or in the same pharmaceutical composition, include, but are not limited to, other compounds which improve a patient's lipid profile, such as (i) HMG-CoA reductase inhibitors, (which are generally statins, including lovastatin, simvastatin, rosuvastatin, pravastatin, fluvastatin, atorvastatin, rivastatin, itavastatin, pitavastatin, and other statins), (ii) bile acid sequestrants (cholestyramine, colestipol, dialkylaminoalkyl derivatives of a cross-linked dextran, Colestid®, LoCholest®, (iii) niacin and related compounds, such as nicotinyl alcohol, nicotinamide, and nicotinic acid or a salt thereof, (iv) PPARy agonists, such as gemfibrozil and fenofibric acid derivatives (fibrates), including clofibrate, fenofibrate, bezafibrate, ciprofibrate, and etofibrate, (v) cholesterol absorption inhibitors, such as stanol esters, beta-sitosterol, sterol glycosides such as tiqueside; and azetidinones, such as ezetimibe, (vi) acyl CoA:cholesterol acyltransferase (ACAT) inhibitors, such as avasimibe and melinamide, and including selective ACAT-1 and ACAT-2 inhibitors and dual inhibitors, (vii) phenolic anti-oxidants, such as probucol, (viii) microsomal triglyceride transfer protein (MTP)/ApoB secretion inhibitors, (ix) anti-oxidant vitamins, such as vitamins C and E and beta carotene, (x) thyromimetics, (xi) LDL receptor inducers, (xii) platelet aggregation inhibitors, for example glycoprotein Ith/IIIa fibrinogen receptor antagonists and aspirin, (xiii) vitamin B12 (also known as cyanocobalamin), (xiv) folic acid or a pharmaceutically acceptable salt or ester thereof, such as the sodium salt and the methylglucamine salt, (xv) FXR and LXR ligands, including both inhibitors and agonists, (xvi) agents that enhance ABCA1 gene expression, and (xvii) ileal bile acid transporters.

Preferred classes of therapeutic compounds that can be used with the DNA and/or protein of the present invention for use in improving a patient's lipid profile (i.e. raising HDL-c and lowering LDL-c) include one or both of statins and cholesterol absorption inhibitors. Particularly preferred are combinations of DNA and/or protein of the present invention with simvastatin, ezetimibe, or both simvastatin and ezetimibe. Also preferred are combinations of DNA and/or protein of the present invention with statins other than simvastatin, such as lovastatin, rosuvastatin, pravastatin, fluvastatin, atorvastatin, rivastatin, itavastatin, and ZD-4522.

Finally DNA and/or protein of the present invention can be used with compounds that are useful for treating other diseases, such as diabetes, hypertension and obesity, as well as other anti-atherosclerostic compounds. Such combinations may be used to treat one or more of such diseases as diabetes, obesity, atherosclerosis, and dyslipidemia, or more than one of the diseases associated with metabolic syndrome. The combinations may exhibit synergistic activity in treating these diseases, allowing for the possibility of administering reduced doses of active ingredients, such as doses that otherwise might be sub-therapeutic.

Examples of other active ingredients that may be administered in combination with DNA and/or protein of the present invention include, but are not limited to, compounds that are primarily anti-diabetic compounds, including:
(a) PPAR gamma agonists and partial agonists, including glitazones and non-glitazones (e.g. pioglitazone, englitazone, MCC-555, rosiglitazone, balaglitazone, netoglitazone, T-131, LY-300512, and LY-818;
(b) biguanides such as metformin and phenformin;
(c) protein tyrosine phosphatase-1B (PTP-1B) inhibitors;
(d) dipeptidyl peptidase IV (DP-IV) inhibitors, including vildagliptin, sitagliptin and saxagliptin;
(e) insulin or insulin mimetics, such as for example insulin lispro, insulin glargine, insulin zinc suspension, and inhaled insulin formulations;
(f) sulfonylureas, such as tolbutamide, glipizide, glimepiride, acetohexamide, chlorpropamide, glibenclamide, and related materials;
(g) α-glucosidase inhibitors (such as acarbose, adiposine; camiglibose; emiglitate; miglitol; voglibose; pradimicin-Q; and salbostatin);
(h) PPARαγ dual agonists, such as muraglitazar, tesaglitazar, farglitazar, and naveglitazar;
(i) PPARδ agonists such as GW501516 and those disclosed in WO97/28149;
(j) glucagon receptor antagonists;
(k) GLP-1; GLP-1 derivatives; GLP-1 analogs, such as exendins, such as for example exenatide (Byetta); and non-peptidyl GLP-1 receptor agonists;
(l) GIP-1; and
(m) Non-sulfonylurea insulin secretagogues, such as the meglitinides (e.g. nateglinide and rapeglinide).

These other active ingredients that may be used in combination with the current invention also include antiobesity compounds, including 5-HT (serotonin) inhibitors, neuropeptide Y5 (NPY5) inhibitors, melanocortin 4 receptor (Mc4r) agonists, cannabinoid receptor 1 (CB-1) antagonists/inverse agonists, and β3 adrenergic receptor agonists. These other active ingredients also include active ingredients that are used to treat inflammatory conditions, such as aspirin, non-steroidal anti-inflammatory drugs, glucocorticoids, azulfidine, and selective cyclooxygenase-2 (COX-2) inhibitors, including etoricoxib, celecoxib, rofecoxib, and Bextra.

Antihypertensive compounds may also be used advantageously in combination therapy with the DNA and/or protein of the present invention. Examples of antihypertensive compounds that may be used with the DNA and/or protein of the present invention include (1) angiotensin II antagonists, such as losartan; (2) angiotensin converting enzyme inhibitors (ACE inhibitors), such as enalapril and captopril; (3) calcium channel blockers such as nifedipine and diltiazam; and (4) endothelian antagonists. Anti-obesity compounds may be administered in combination with the compounds of this invention, including: (1) growth hormone secretagogues and growth hormone secretagogue receptor agonists/antagonists, such as NN703, hexarelin, and MK-0677; (2) protein tyrosine phosphatase-1B (PTP-1B) inhibitors; (3) cannabinoid receptor ligands, such as cannabinoid CB1 receptor antagonists or inverse agonists, such as rimonabant (Sanofi Synthelabo), AMT-251, and SR-14778 and SR 141716A (Sanofi Synthelabo), SLV-319 (Solvay), BAY 65-2520 (Bayer); (4) anti-obesity serotonergic agents, such as fenfluramine, dexfenfluramine, phentermine, and sibutramine; (5) β3-adrenoreceptor agonists, such as AD9677/TAK677 (Dainippon/Takeda), CL-316,243, SB 418790, BRL-37344, L-796568, BMS-196085, BRL-35135A, CGP12177A, BTA-243, Trecadrine, Zeneca D7114, and SR 59119A; (6) pancreatic lipase inhibitors, such as orlistat (Xenical®), Triton WR1339, RHC80267, lipstatin, tetrahydrolipstatin, teasaponin, and diethylumbelliferyl phosphate; (7) neuropeptide Y1 antagonists, such as BIBP3226, J-115814, BIBO 3304, LY-357897, CP-671906, and GI-264879A; (8) neuropeptide Y5 antagonists, such as GW-569180A, GW-594884A, GW-587081×, GW-548118×, FR226928, FR 240662, FR252384, 1229U91, GI-264879A, CGP71683A, LY-377897, PD-160170, SR-120562A, SR-120819A and JCF-104; (9) melanin-concentrating hormone (MCH) receptor antagonists; (10) melanin-concentrating hormone 1 receptor (MCH1R) antagonists, such as T-226296 (Takeda); (11) melanin-concentrating hormone 2 receptor (MCH2R) agonist/antagonists; (12) orexin-1 receptor antagonists, such as SB-334867-A; (13) melanocortin agonists, such as Melanotan II; (14) other Mc4r (melanocortin 4 receptor) agonists, such as CHIR86036 (Chiron), ME-10142, and ME-10145 (Melacure), CHIR86036 (Chiron); PT-141, and PT-14 (Palatin); (15) 5HT-2 agonists; (16) 5HT2C (serotonin receptor 2C) agonists, such as BVT933, DPCA37215, WAY161503, and R-1065; (17) galanin antagonists; (18) CCK agonists; (19) CCK-A (cholecystokinin-A) agonists, such as AR-R 15849, GI 181771, JMV-180, A-71378, A-71623 and SR146131; (20) GLP-1 agonists; (21) corticotropin-releasing hormone agonists; (22) histamine receptor-3 (H3) modulators; (23) histamine receptor-3 (H3) antagonists/inverse agonists, such as hioperamide, 3-(1H-imidazol-4-yl)propyl N-(4-pentenyl)carbamate, clobenpropit, iodophenpropit, imoproxifan, and GT2394 (Gliatech); (24) β-hydroxy steroid dehydrogenase-1 inhibitors (11β-HSD-1 inhibitors), such as BVT 3498 and, BVT 2733, (25) PDE (phosphodiesterase) inhibitors, such as theophylline, pentoxifylline, zaprinast, sildenafil, aminone, milrinone, cilostamide, rolipram, and cilomilast; (26) phosphodiesterase-3B (PDE3B) inhibitors; (27) NE (norepinephrine) transport inhibitors, such as GW 320659, despiramine, talsupram, and nomifensine; (28) ghrelin receptor antagonists; (29) leptin, including recombinant human leptin (PEG-OB, Hoffman La Roche) and recombinant methionyl human leptin (Amgen); (30) leptin derivatives; (31) BRS3 (bombesin receptor subtype 3) agonists such as [D-Phe6,beta-Ala11,Phe13,Nle14]Bn(6-14) and [D-Phe6,Phe13]Bn(6-13)propylamide; (32) CNTF (Ciliary neurotrophic factors), such as GI-181771 (Glaxo-SmithKline), SR146131 (Sanofi Synthelabo), butabindide, PD170,292, and PD 149164 (Pfizer); (33) CNTF derivatives, such as axokine (Regeneron); (34) monoamine reuptake inhibitors, such as sibutramine; (35) UCP-1 (uncoupling protein-1, 2, or 3) activators, such as phytanic acid, 4-[(E)-2-(5,6,7,8-tetrahydro-5,5,8,8-tetramethyl-2-napthalenyl)-1-propenyl]benzoic acid (TTNPB), and retinoic acid; (36) thyroid hormone β agonists, such as KB-2611 (KaroBioBMS); (37) FAS (fatty acid synthase) inhibitors, such as Cerulenin and C75; (38) DGAT1 (diacylglycerol acyltransferase 1) inhibitors; (39) DGAT2 (diacylglycerol acyltransferase 2) inhibitors; (40) ACC2 (acetyl-CoA carboxylase-2) inhibitors; (41) glucocorticoid antagonists; (42) acyl-estrogens, such as oleoyl-estrone; (43) dicarboxylate transporter inhibitors; (44) peptide YY, PYY 3-36, peptide YY analogs, derivatives, and fragments such as BIM-43073D, BIM-43004C, (45) Neuropeptide Y2 (NPY2) receptor agonists such NPY3-36, N acetyl [Leu(28,31)] NPY 24-36, TASP-V, and cyclo-(28/32)-Ac-[Lys28-Glu32]-(25-36)-pNPY; (46) Neuropeptide Y4 (NPY4) agonists such as pancreatic peptide (PP); (47) Neuropeptide Y1 (NPY1) antagonists such as BIBP3226, J-115814, BIBO 3304, LY-357897, CP-671906, and GI-264879A; (48) Opioid antagonists, such as nalmefene (Revex®), 3-methoxynaltrexone, naloxone, and naltrexone; (49) glucose transporter inhibitors; (50) phosphate transporter inhibitors; (51) 5-HT (serotonin) inhibitors; (52) beta-blockers; (53) Neurokinin-1 receptor antagonists (NK-1 antagonists); (54) clobenzorex; (55) cloforex; (56) clominorex; (57) clortermine; (58) cyclexedrine; (59) dextroamphetamine; (60) diphemethoxidine, (61) N-ethylamphetamine; (62) fenbutrazate; (63) fenisorex; (64) fenproporex; (65) fludorex; (66) fluminorex; (67) furfurylmethylamphetamine; (68) levamfetamine; (69) levophacetoperane; (70) mefenorex; (71) metamfepramone; (72) methamphetamine; (73) norpseudoephedrine; (74) pentorex; (75) phendimetrazine; (76) phenmetrazine; (77) picilorex; (78) phytopharm 57; (79) zonisamide, (80) a minorex; (81) amphechloral; (82) amphetamine; (83) benzphetamine; and (84) chlorphentermine.

The combination therapies described above which use the DNA and/or protein of the present invention may also be useful in the treatment of the metabolic syndrome. According to one widely used definition, a patient having metabolic syndrome is characterized as having three or more symptoms selected from the following group of five symptoms: (1) abdominal obesity; (2) hypertriglyceridemia; (3) low high-density lipoprotein cholesterol (HDL); (4) high blood pressure; and (5) elevated fasting glucose, which may be in the range characteristic of Type 2 diabetes if the patient is also diabetic. Each of these symptoms is defined clinically in the recently released Third Report of the National Cholesterol Education Program Expert Panel on Detection, Evaluation and Treatment of High Blood Cholesterol in Adults (Adult Treatment Panel III, or ATP III), National Institutes of Health, 2001, NIH Publication No. 01-3670. Patients with metabolic syndrome have an increased risk of developing the macrovascular and microvascular complications that are listed above, including atherosclerosis and coronary heart disease. The combinations described above may ameliorate more than one symptom of metabolic syndrome concurrently (e.g. two symptoms, three symptoms, four symptoms, or all five of the symptoms).

### FIGURES

FIG. 1 shows a schematic description of the immunization protocols. Specifically the figure shows the chronological order of when the mice models were injected with hPCSKP DNA or protein, the control DNA or protein and the CpG adjuvant. The scheme shows when samples were extracted from the mice in order to test for an immune response.

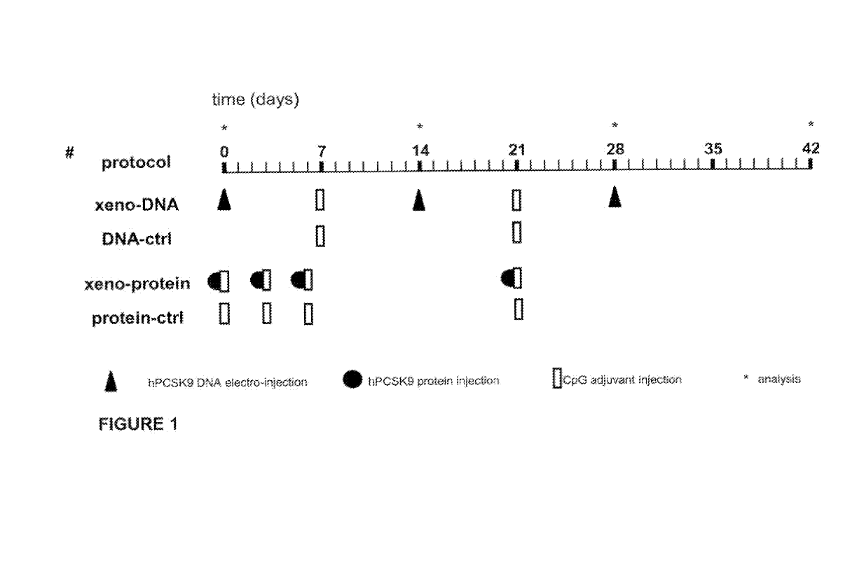

FIG. 2 shows that immunization with hPCSK9 elicits humoral response against the mouse endogenous gene product. In order to test this anti-mPCSK9 antibody titers were measured by ELISA in the serum from mice immunized with protein (empty squares; n=10) DNA (filled triangles; n=10). Control-protein and control-DNA showed a low and comparable reactivity in this as well as in the other reported experiment. We therefore referred to them as a unique group indicated a “negative-ctrl” (empty circles; n=15). Dashed lines indicate the dilution interval used as described in the Material and Methods. Data falling outside this interval were reported as the minimal or maximal dilution, as appropriate. FIG. 2 illustrates that a measurable humeral response was shown in the mice model when immunized with hPCSK9.

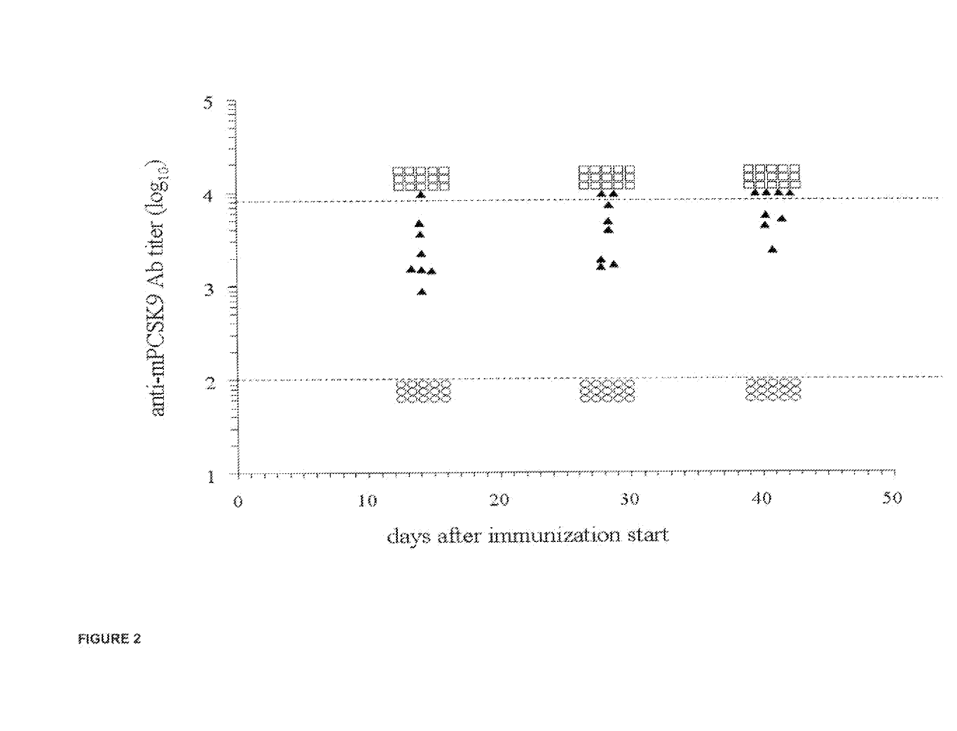

FIG. 3 shows lipoprotein levels upon immunization. The four charts show that total cholesterol (A), HDL-c (B), LDL-c (C) and triglycerids (D) were monitored every 14 days after the immunization program started. The display items show the analysis of sera of mice immunized according to protein (filled triangles; n=15), DNA (filled squares; n=15) and negative-ctrl (empty circles; n=10). Chart D shows that the triglycerides levels of the experimental groups remained very similar. Chart C shows that there is a marked decrease in LDL-c when the mice were immunized with protein or DNA after 14 days.

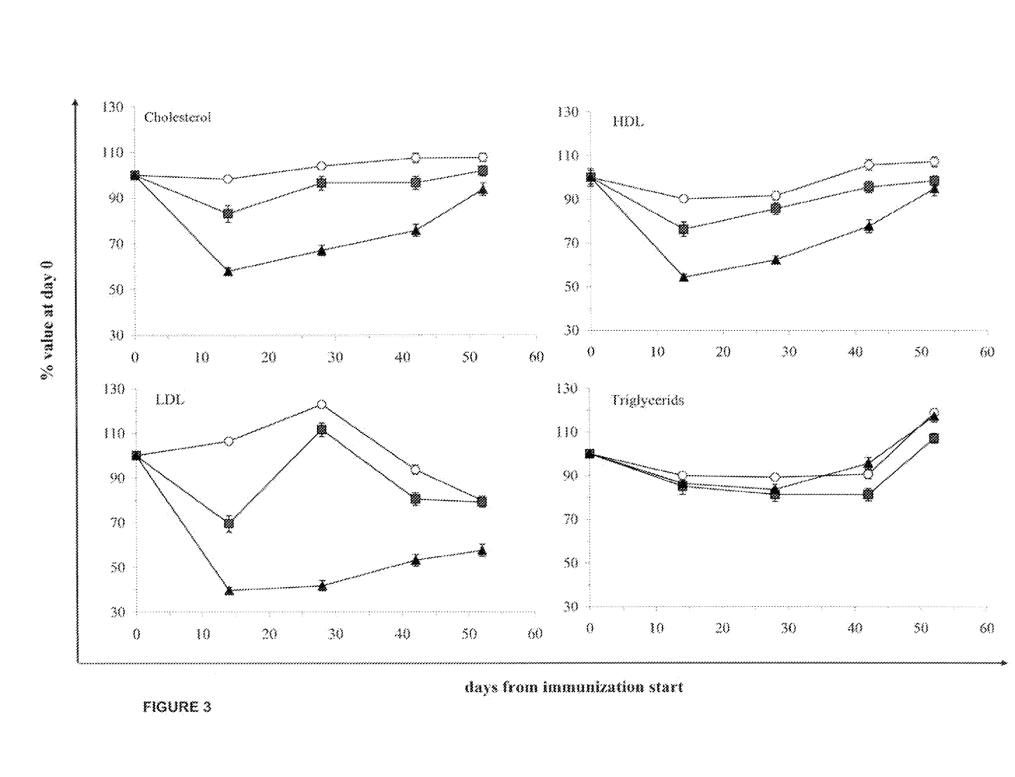

FIG. 4 shows a correlation between LDL-c levels, anti-mouse PCSK9 antibody titers and plasma mPCSK9 concentration in immunized mice. Correlation plot of circulating LDL-c levels b anti-mPCSK 9 Ab titers measured at day 14 after immunization start according to DNA (filled triangles; n=10); protein (empty square; n=10) and negative-ctrl (empty circles, n=10). Dashed lines indicate the dilution interval. Data falling outside this dilution interval were reported as the minimal or maximal dilution, as appropriate. Spearman rank test was used to assess the correlation among LDL-c and the anti-mPCSK9 antibodies in immunized and in control mice (ρ=−0.79; p=1.4e-9). Circulating mPCSK9 levels at day 14 in mice immunized with the hPCSK9 protein and non-immunized mice

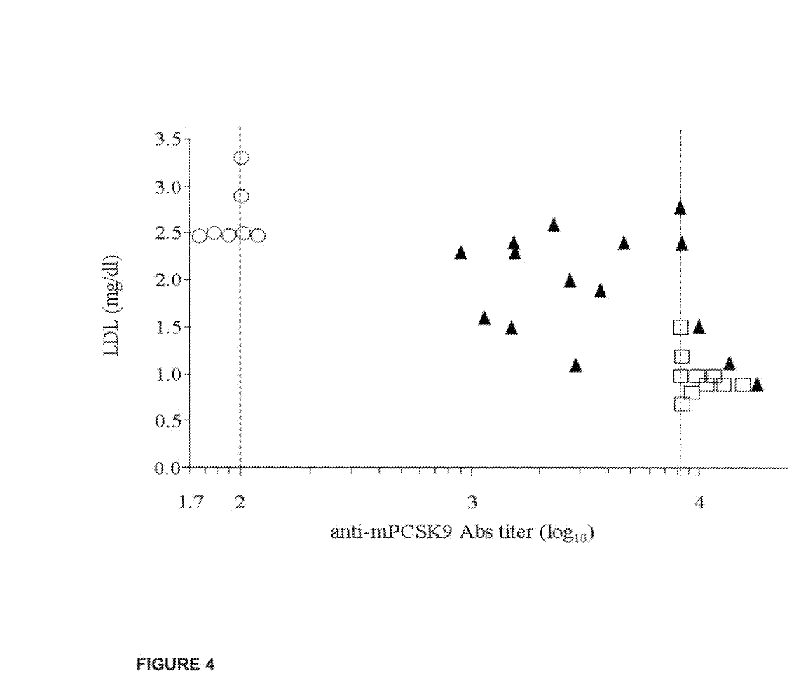

FIG. 5 shows levels and cellular distribution of LDLR in livers of immunized mice. (A) Liver extracts from mice immunized performed according to protein (samples 6-10) or control-protein (samples 1-5) protocol were separated by SDS-PAGE with a molecular weight marker (M) and blotted onto a nitro-cellulose filter. Following incubation with labeled anti-LDLR or anti-tubulin antibody, the bands corresponding to LDLR or α-tubulin proteins were visualized by autoradiography. The LDLR signal was measured by densitometric scanning and normalized on the corresponding α-tubulin signal. (B) Shows cellular distribution of LDLR in immunized mice. Immuno-histochemistry analysis was performed on liver samples obtained from the same mice immunized with protein or control-protocol using x-labeled anti-LDLR.

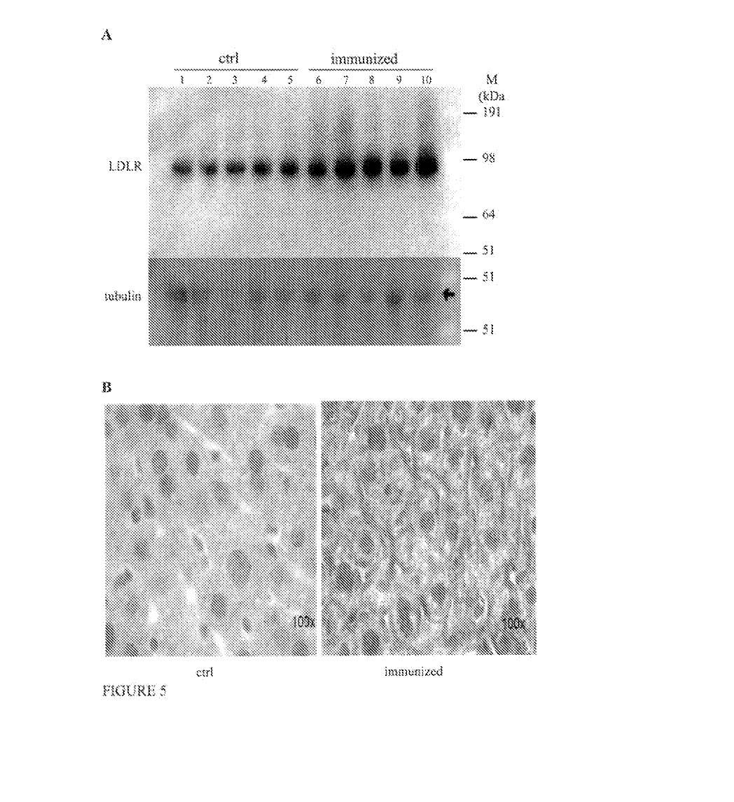

FIG. 6 shows the levels of mPCSK9 ELISA. (A) Standard curve for mPCSK9 ELISA using Mab A1 and Fab A08 (see Example 6). Purified mPCSK9 was used as standard. The sensitivity of the assay is ˜100 μM. (B) Plasma matrix effect on mPCSK9 ELISA (see Example 6). Mouse plasma were serial diluted and PCSK9 level were measured by ELISA.

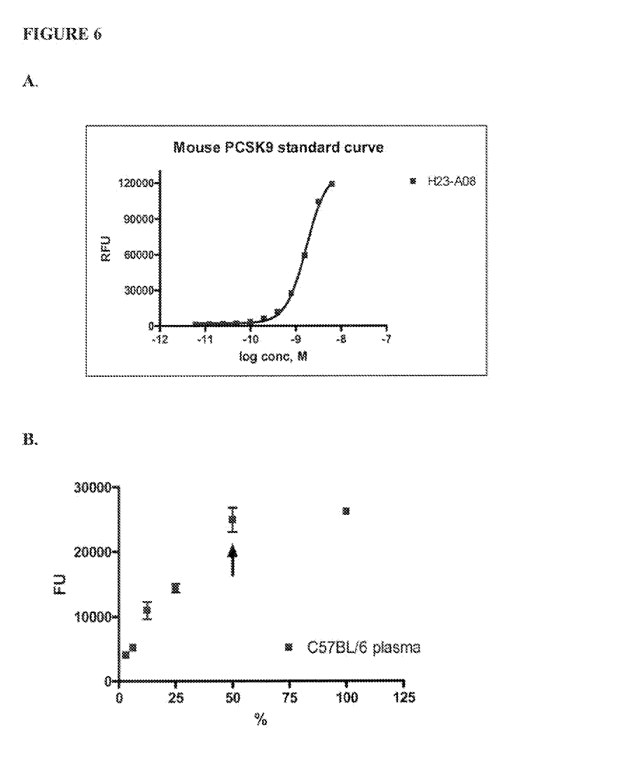

The following examples illustrate, but do not limit the invention.

### EXAMPLES

### Example 1

### Immunogens

Full-length hPCSK9 and mPCSK9 were amplified from human or mouse fetal liver, respectively, and cloned under the transcriptional control of human Cytomegalovirus (CMV) promoter in pVIJ expression plasmid. A pVIJ_hPCSK9 and pVIJ_mPCSK9 derivatives with C-terminal V5 and 6-His epitope tags were expressed in stably transfected HEK293 cell lines and purified as previously described.

### Example 2

### Animal Studies

Female BALB/c and C57/bl6 mice were bred under specific pathogen-free conditions by Charles River Breeding Laboratories (Calco, Como, Italy). In all operations, mice were treated in accordance with European guidelines Animals were maintained in standard conditions under a 12-hour light-dark cycle, provided irradiated food (Mucedola, Settimo Milanese, Italy) and water ad libitum. Mice were fully anesthetized with ketamine (Merial Italia, Milano, Italy) at 100 mg/kg of body weight and xylazine (BIO 98, Bologna, Italy) at 5.2 mg/kg. The immunization experiments were all performed at the Istituto di Ricerche di Biologia Molecolare, which has been awarded full AAALAC accreditation.

### Example 3

### Immunization Protocol

Mice were electro-injected intramuscularly 3 times at 2 week intervals (week 0, 2, 4) with pVIJ_hPCSK9 and CpG adjuvant (50 μg/mouse/injection each) 3 times at 2 weeks interval (week 1, 3, 5). The CpG used in this study was a 20-mer (5′-TCCATGACGTTCCTGACGTT-3′) with a nuclease-resistant phosphorothioate backbone, which contains two CpG motifs with known immuno-stimulatory effects on the murine immune response. Control mice were injected only with CpG (50 μg/mouse/injection) 3 times at 2 week intervals (at day 7, 21 and 35). The hPCSK9 protein formulated with CpG (100 μg protein and 50 μg adjuvant) was injected subcutaneously at the base of the tail on day 0, 3, 6. Control mice received only CpG.

### Example 4

### Measure of Clinical Chemical Parameters

Peripheral blood was collected and direct HDL-c, direct LDL-c and total cholesterol were measured using the ADVIA 1200 IMS (Bayer Healthcare, Terrytown, N.Y.).

### Example 5

### ELISA Detection of Plasma Anti-hPCSK9 and Anti-mPCSK9 Serum Antibodies

Multiwell Maxisorp ELISA plates (Nunc, Roskild, Denmark) were coated overnight with PCSK9 protein at a concentration of 5 μg/mL in 50 mM NaHCO₃ (pH 9.6). Plates were then briefly rinsed with washing buffer (0.05% Tween-20 in PBB; PBST) and incubated for 1 hr at 37° C. with blocking buffer (3% non-fat dry milk/0.05% Tween-20 in PBS; PBSMT). Serial dilutions of pre-immune or immune sera in PBST (ranging from 1:100 to 1/8,100) were added to the wells and incubated for 2 hrs at room temperature. Plates were washed and incubated with mAb anti-mouse IgG Fc-specific, AP-conjugated (Sigma-Aldrich Inc., St. Louis, Mo.) for 60 min at RT and alkaline phosphatase activity detected by incubation with AP substrate solution (Sigma-Aldrich Inc., St. Louis, Mo.) in 10% diethanolamine/0.5 mM MgCl₂.

Titers of anti-PCSK9 antibodies in the serum of immunized mice were computed as follows. Experimental data were acquired as (A₄₀₅ₙₘ-A₄₆₀ₙₘ) for each dilution of each sample. For each animal, pre-bleeds were included in duplicates at a 1:100 fold dilution together with the serial dilutions. We observed some variability in the pre-bleeds (A₄₀₅ₙₘ-A₄₆₀ₙₘ) between plates, but not within each plate. The baseline was therefore determined as the mean+3 standard deviations of the pre-bleed (A₄₀₅ₙₘ-A₄₆₀ₙₘ) for each plate, individually. Data referring to the same sample were fitted by a monotone Hermite spline (49). Titers were defined as the dilution at which the base line intersects the (A₄₀₅ₙₘ-A₄₆₀ₙₘ). When this intersection fell outside the dilution interval, titers were reported as the minimal or maximal dilutions, as appropriate.

### Example 6

### Determination of Plasma mPCSK9 Concentration

High binding 4HBX plates (ThermoLabsystems, Helsinki, Finland) were coated overnight at 4° C. with 50 μl of 10 μg/ml of anti-mPCSK9 A1 antibody. Next day, the wells were first blocked for 1 hr at room temperature with 250 μl of blocking solution (1% BSA (KPL) in TBS (BIORAD, Hercules, Calif.) and 0.05% Tween-20) and then washed in a plate-washer with washing buffer (KPL). Purified mPCSK9 protein diluted in 1% BSA in PBS (as standard) or mice plasma were added to the wells and were incubated at 37° C. for 2 hrs followed by a washing step. Then, 100 μl of 1 μg/ml biotinylated anti-mPCSK9 Fab, A2, was added to the plate and, after an additional washing step, 75 μl of 1:1,000 Streptavidin/Europium (Perkin Elmer, Waltham, Mass.) were added. Plates were incubated at room temperature for 20 min, followed by a last washing step and the addition of 100 μl of DELFIA enhancer (Perkin Elmer, Waltham, Mass.). After 1 hr the plates were read with a Europium reader. The sensitivity threshold of the assay is ˜100 μM. The precision of the ELISA was assessed using C57BL/6 mouse plasma samples. The intra-assay imprecision (% CV) was less than 10% (n=31). Serial dilution of mouse plasma samples showed that serum or plasma tolerance of the assay is approximately 50%.

### Example 7

### Western Blot Analysis of Liver LDLR

Liver crude protein extracts were prepared using RIPA Lysis buffer 500 μl per mg tissue (Santa Cruz Biotechnology Inc., Santa Cruz, Calif.) with PMSF, sodium orthovanadate and proteinase inhibitors. NuPAGE 4-12% Bis-Tris Gels (Life Technologies, Carlsband, Calif.) were loaded with 50 μg protein per lane. Blotted proteins were developed using mLDLR goat IgG (R&D Systems, Minneapolis, Minn.), 1:1000 diluted in primary Ab diluent buffer. Tubulin antibody (Sigma, St Louis, Mo.) T-5168 mouse mAb, (1:5,000) was used to normalize data. Quantification of bands on X-Ray was made with IMAGE READER LAS 3000 (Fujifilm, Tokyo, Japan).

### Example 8

### Immunohistochemical Staining for Mouse LDLR

Immunohistochemistry was performed essentially as described in (50). Briefly, tissues were fixed in 10% buffered formalin and embedded in paraffin. 10 μm microtome sections were cleared in xylol and re-hydrated; the unmasking procedure was carried out by immerging the samples in DAKO antigen retrieve solution 10× (DAKO, Glostrup, Denmark) at 99° C. for 40 min; after rinsing in PBS, the sections were covered with blocking solution (15 μl goat serum in 1 ml PBS), for 30 min at RT. Without additionally rinse the sections were covered with Rabbit polyclonal anti-LDL receptor (Abcam Inc., Cambridge, UK), incubated 1 hour at RT and then rinsed in PBS and incubated with Goat anti-rabbit IgG Peroxidase conjugated antibody (Sigma-Aldrich Inc., St. Louis, Mo.) at the dilution of 1:200 for additional 60 min. After rinsing in PBS, the sections were stained with the diaminobenzidine staining kit (Vector Laboratories Inc., Burlingame, Calif.) and nuclei were stained with hematoxylin. The sections were dehydrated and mounted with Entellan (Merck KGaA, Darmstadt).

1.-14. (canceled)

15. An isolated nucleic acid selected from the group consisting of:
(a) a nucleic acid encoding a xenographic PCSK9 or mutated xengraphic PCSK9;
(b) a nucleic acid encoding a human-nonhuman chimeric PCSK9 or mutated human-nonhuman chimeric PCSK9; and
(c) a nucleic acid encoding an immunologically active fragment of (a) or (b).

16. The nucleic acid of claim 15, wherein the xenographic PCSK9 or nonhuman PCSK9 is from mouse or canine.

17. An isolated polypeptide encoded by the nucleic acid molecule of claim 15.

18. The polypeptide of claim 17, wherein the polypeptide comprises a portion of the polypeptide that binds to the LDL-receptor.

19. A pharmaceutical composition comprising a polypeptide of claim 17 and a pharmaceutically acceptable carrier.

20. The pharmaceutical composition of claim 19 further comprising an adjuvant.

21. The pharmaceutical composition of claim 20, wherein the adjuvant is an immunostimulatory oligonucleotide comprising unmethylated CpG dinucleotides.

22. A method of treating a disorder associated with high LDL-levels in patient comprising administering to a patient in need thereof the composition of claim 20.

23. The method of claim 22, wherein the disorder is selected from the group consisting of cardiovascular disease and atherosclerosis.
